(19) United States
(12) Patent Application Publication
(10) Pub. No.: US 2025/0120612 A1
(43) Pub. Date: Apr. 17, 2025

(54) SYSTEM AND METHOD FOR DETECTING DIAPER CHANGES IN PATIENTS

(71) Applicant: Tochtech Technologies Ltd., Delta (CA)

(72) Inventors: Wei Fang, Langley (CA); Jing Yang, Richmond (CA)

(21) Appl. No.: 18/916,275

(22) Filed: Oct. 15, 2024

Related U.S. Application Data
(60) Provisional application No. 63/590,267, filed on Oct. 13, 2023.

(51) Int. Cl. A61B 5/11; A61B 5/00

(52) U.S. Cl. CPC A61B 5/1115; A61B 5/6887

(57) ABSTRACT

A system for detecting possible diaper changes in bedridden patients includes a load cell and a computer configured to receive data from the load cell. The load cell is placed under a bed and is configured to detect changes in the forces applied onto the load cell. These changes in forces may include changes in the apparent bed weight and an indicator of detected movement strength. The data from the load cell may be processed by the computer to determine whether a diaper change has likely occurred. The computer may be configured to apply machine learning techniques to compare data patterns from the load cell with previous data corresponding to known diaper changes.

### CROSS-REFERENCE TO RELATED APPLICATIONS

This application claims the benefit of U.S. Provisional Patent Application No. 63/590,267 filed Oct. 13, 2023, the contents of which are hereby incorporated by reference.

### TECHNICAL FIELD

The present disclosure relates to systems and methods for detecting when a diaper change has occurred. In particular, the present disclosure relates to systems and methods for detecting when nursing staff perform diaper changes for bedridden patients.

### BACKGROUND

In care facilities, nursing homes, hospitals, and other similar settings, many of the patients may require assistance with personal hygiene, including, for example, requiring the use of adult diapers. Such diapers and other personal hygiene items may need to be changed regularly in order to maintain a certain level of personal hygiene. It is therefore helpful to be able to track when a patient's adult diaper may have been changed.

### SUMMARY

In accordance with one aspect, a system for detecting possible diaper changes in bedridden patients comprises a load cell and a computer configured to receive data from the load cell. The load cell is placed under a bed and is configured to detect changes in the forces applied onto the load cell. These changes in forces may include changes in the apparent bed weight and an indicator of detected movement strength. The data from the load cell may be processed by the computer to determine whether a diaper change has likely occurred. The computer may be configured to apply machine learning techniques to compare data patterns from the load cell with previous data corresponding to known diaper changes.

In one aspect, a method for detecting a possible changing of a diaper of a patient in a bed comprising one or more bed supports comprises the steps of: providing a device comprising one or more load cells and a transmitter; placing the device under one of the one or more bed supports; sensing, by the one or more load cells, of a magnitude of a force exerted on the one or more load cells at each of a plurality of times over a period of time, the magnitude of the force corresponding to an apparent bed weight; transmitting, by the transmitter to a server, data sensed by the one or more load cells, the data comprising information corresponding to a magnitude of the force exerted on the one or more load cells at each of the plurality of times; storing, by the server, of the data to generate a record comprising a temporal history of the magnitude of the force exerted on the one or more load cells over the period of time; configuring the server to recognize a pattern indicative of the possible changing of the diaper of the patient based, at least in part, on changes in the apparent bed weight; determining, by the server whether the possible changing of the diaper of the patient has occurred, based, at least in part on whether the temporal history comprises the pattern; and recording, by the server, a time in the period of time when the temporal history comprises the pattern.

In another aspect, the method further comprises the step of sensing, by the one or more load cells, of a degree of vibration of the force exerted on the one or more load cells at each of the plurality of times over the period of time, the degree of vibration of the force corresponding to a movement strength.

In yet another aspect, the step of configuring the server to recognize the pattern indicative of the possible changing of the diaper of the patient is also based, at least in part, on changes in the movement strength.

In still yet another aspect, the pattern comprises at least four phases, the at least four phases corresponding to (1) rolling of the patient away to one side; (2) replacing the diaper of the patient; (3) rolling the patient back to another side to fasten the diaper; and (4) rolling the patient back to a resting position.

In a further aspect, the step of configuring the server to recognize the pattern indicative of the possible changing of the diaper of the patient based, at least in part, on changes in the apparent bed weight comprises using shapelet learning techniques.

In still a further aspect, the step of configuring the server to recognize the pattern indicative of the possible changing of the diaper of the patient also based, at least in part, on changes in the movement strength, comprises using shapelet learning techniques.

In another aspect, the method further comprises the step of determining, by the server, whether the patient is likely in the bed at one of the plurality of times, based, at least in part, on the apparent bed weight.

In still another aspect, the method further comprises the step of generating, by the server, of an alert if the server determines that the possible changing of the diaper for the patient has not occurred within a pre-set length of time.

In still yet another aspect, the step of storing, by the server, of the data to generate the record comprising the temporal history of the magnitude of the force exerted on the one or more load cells over the period of time comprises storing of the data in a database in communications with the server.

In a further aspect, the step of transmitting, by the transmitter to the server, data sensed by the one or more load cells occurs approximately every five minutes when the magnitude of the force exerted on the one or more load cells has not changed within a first pre-set interval of time.

In still a further aspect, the step of transmitting, by the transmitter to the server, data sensed by the one or more load cell occurs approximately every second when the magnitude of the force exerted on the one or more load cells has changed within a second pre-set interval of time.

In still yet a further aspect, the step of storing, by the server, of the data to generate the record comprising the temporal history of the magnitude of the force exerted on the one or more load cells over the period of time further comprises processing, by the server, of the data by applying one or more of the following: segmentation of the data, interpolation of the data, noise reduction of the data, normalization of the data, and feature extraction of the data.

In another aspect, the step of determining, by the server whether the possible changing of the diaper of the patient has occurred, is also based, at least in part on whether the changes in the apparent bed weight are above a first pre-set threshold.

In still another aspect, the step of determining, by the server whether the possible changing of the diaper of the patient has occurred, is also based, at least in part on whether the changes in the movement strength are above a second pre-set threshold.

The foregoing was intended as a summary only and of only some of the aspects of the invention. It was not intended to define the limits or requirements of the invention. Other aspects of the invention will be appreciated by reference to the detailed description of the preferred embodiments.

### BRIEF DESCRIPTION OF THE DRAWINGS

The invention will be described by reference to the detailed description of the embodiments and to the drawings thereof in which:

FIG. 1 depicts one embodiment of the system;

FIG. 2 is a partial magnified view of the system of FIG. 1;

FIG. 3 depicts data from the load cell for a non-bedridden patient;

FIG. 4 depicts data from the load cell for a bedridden patient;

FIG. 5 depicts data from the load cell for a bedridden patient;

FIG. 6 is a flow chart depicting the process flow of the system;

FIG. 7 is a system diagram of some of the aspects of the system;

FIG. 8 depicts raw irregular interval data from the load cells;

FIG. 9 depicts data with interpolation applied to generate constant interval data;

FIG. 10 depicts data with noise reduction applied to generate more smoothed data;

FIG. 11 depicts data with normalization applied;

FIG. 12 depicts a possible machine learning detection engine data processing workflow;

FIG. 13 depicts a possible shapelet discovery workflow; and

FIG. 14 depicts a plot of loss over total training iterations for the learning shapelets approach.

### DETAILED DESCRIPTION

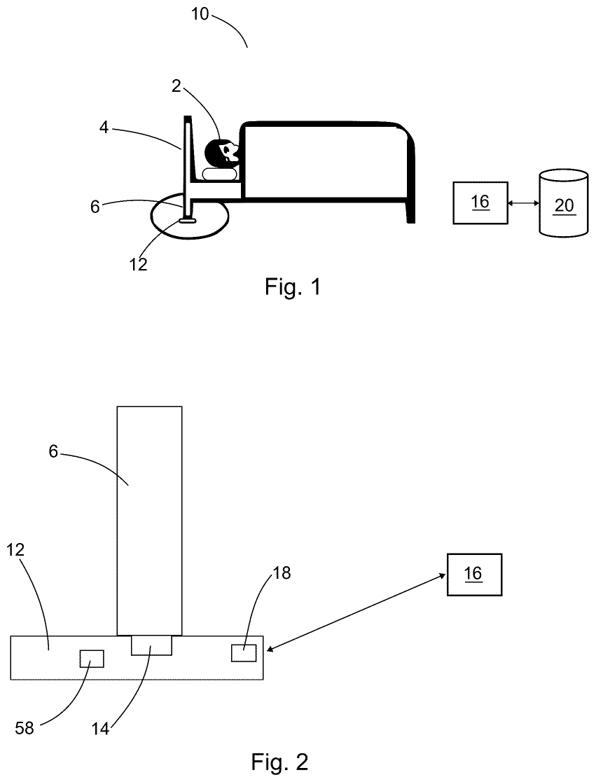

Referring to FIGS. 1 and 2, in some embodiments, a system 10 for detecting diaper changes for a patent 2 in a bed 4 comprises one or more devices 12 that may be placed under the bed 4. For example, the bed 4 may one or more bed supports 6 (such as legs or the like). One of the devices 12 may be placed under one or more of the bed supports 6. In some embodiments, the system 10 may comprise one of the devices 12, with the device 12 placed under one of the bed supports 6, as shown in FIG. 1. It is understood that more than one of the devices 12 may be provided in the system 10, with each one of the devices 12 placed under one of the bed supports 6. In embodiments where more than one of the devices 12 are provided, by placing the devices 12 under different ones of the bed supports 6, the devices 12 may be placed under different portions or areas of the bed 4. For example, the bed supports 6 may be located proximate to the corners of the bed 4. Accordingly, the devices 10 may be located proximate to the corners of the bed 4.

Each of the devices 12 comprises one or more load cells 14. The load cells 14 are configured to convert a force (such as tension, compression, pressure, torque, etc.) into an electrical signal. In embodiments where the device 12 comprise more than one of the load cells 14, the electrical signals from the load cells 14 may be averaged so that a single electrical signal is generated.

The system 10 may further comprise a server 16 that is configured to receive data from the one or more devices 12. For example, the devices 12 may further comprise a transmitter 18 that is configured to communicate with the server 16 to transmit the electrical signal(s) generated by the load cells 14. The transmitter 18 may be configured to communicate with the server 16 either wirelessly or in a wired manner. For example, the transmitter 18 may use Wi-Fi or some other suitable wireless communications protocol to communicate with the server 16.

The patient 2 may be bedridden (e.g. generally confined to the bed 4) and may require assistance in maintaining personal hygiene. For example, the patient 2 may wear a diaper, which may be an adult diaper or some other personal hygiene item. The staff at the facility where the patient 2 is located may be required to regularly change the diaper of the patient 2 in order to maintain a level of personal hygiene for the patient 2.

The typical steps involved in changing the diaper for the patient 2 on the bed 4 may be as follows:
1. Rolling the patient 2 onto one side of the patient 2;
2. Unfastening the potentially soiled diaper;
3. Putting on a new diaper;
4. Rolling the patient 2 onto the other side of the patient 2;
5. Fastening the new diaper; and
6. Returning the patient 2 back to a normal sleep position.

Referring again to FIGS. 1 and 2, the load cells 14 in the devices 12 may be configured to detect the forces applied to the load cells 14 and their relative magnitude, converting them into electrical signals. By monitoring the electrical signals generated by the load cells 14 and their changes over time, it may be possible to detect changes in the forces applied to the load cells 14, which may be indicative of possible movement by the patient on the bed 4. For example, the electrical signals generated from the load cells 14 may be used to detect changes in an apparent bed weight 50 of the bed 4. This change in the apparent bed weight 50 of the bed 4 may be due to a center of gravity of the bed 4 and the patient 2 shifting when the patient 2 turns in the bed 4 (e.g. either away from the device 12 or towards the device 12, when the system 10 comprises one of the devices 12). The load cells 14 may also be configured to detect a relative magnitude of a movement strength 52.

In some embodiments, the load cells 14 may be configured to detect a relative magnitude of the movement strength 52 exerted on the load cells 14 by detecting a level of vibration of the force exerted on the load cells 14. The level of vibration may be measured as the degree of weight change over a given time (e.g. milligram weight change in a millisecond). The movement strength 52 may be determined, at least in part, based on the rate of the weight change, its frequency, and/or its duration.

The load cells 14 are configured to detect the forces applied to the device 12 on which the load cells 14 are located and to convert the forces into electrical signals. The electrical signals may then be transmitted by the transmitter 18 from the device 12 to the server 16 for processing. In some embodiments, the server 16 may apply one or more filters (e.g. low pass filters, band pass filters, etc.) to the electrical signals received from the devices 12. For example, a low pass filter may be applied to isolate data relating to changes to the apparent bed weight 50 of the bed 4, as detected by the load cells 14. Similarly, band pass filters may be applied to isolate data relating to the movement strength 52 and/or vital signs (e.g. heart rate, breathing rate) of the patient 2. The data regarding the apparent bed weight 50 of the bed 4 and the data regarding the movement strength 52 may be used by the server 16 to determine whether a likely diaper change 54 has occurred. In some embodiments, machine learning techniques may also be applied by the server 16 to attempt to further improve the detection and determination process. The data received by the server 16 from the device 12 along with the time that the data was taken or received may be stored in a database 20 that is in communications with the server 16.

In some embodiments, the device 12 may further comprise a controller 58 that controls the frequency or how often data is transmitted by the transmitter 18 to the server 16. For example, the controller 58 may be configured such that if the load cells 14 do not detect any change in the apparent bed weight 50 or the change in the apparent bed weight 50 is less than 1 kg, the data regarding the apparent bed weight 50 and the movement strength 52 may be transmitted by the transmitter 18 every 5 minutes. The controller 58 may be further configured such that if the load cells 14 detects a change in the apparent bed weight 50 of greater than 1 kg, the data regarding the apparent bed weight 50 and the movement strength 52 may be transmitted by the transmitter 18 every second. By controlling and adjusting the frequency or how often data is transmitted by the transmitter 18, the amount of processing required by the server 16 is reduced.

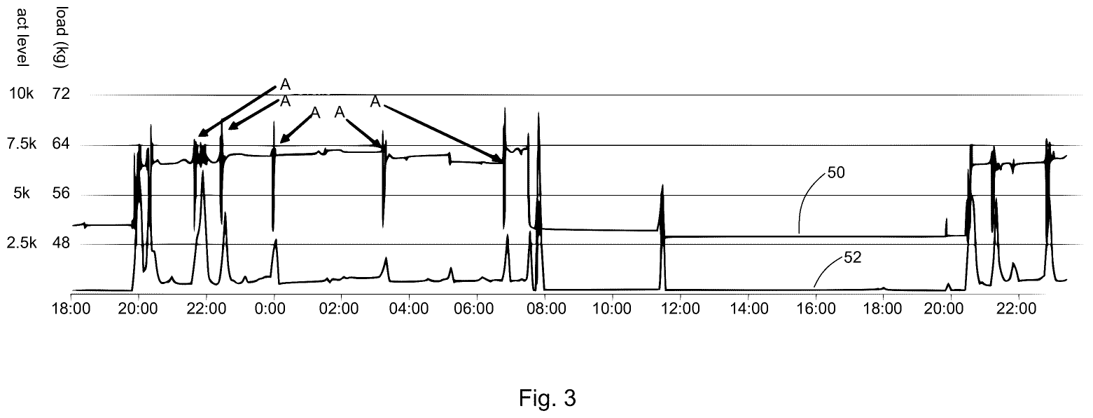

FIG. 3 depicts sample sensor data from the load cells 14 taken over a period of time from the bed 4 for a person that is not bedridden (i.e. the person is able to roll and/or reposition himself or herself regularly and without assistance). Such a person would also not require assistance with diaper changes. The upper line in FIG. 3 is a measure of the apparent bed weight 50 over time, while the lower line is a measure of the movement strength 52. The person would have got into the bed 4 at approximately 20:00 (corresponding to the increase in the apparent bed weight 50 at approximately 20:00) and left the bed at approximately 8:00 (corresponding to the decrease in the apparent bed weight 50 at approximately 8:00). The peaks A in the upper line in FIG. 3 between 20:00 and 8:00 (e.g. at approximately 21:30, 22:30, 24:00, 3:00, and 7:00 may be indicative of natural self-repositioning events carried out by the person during the night.

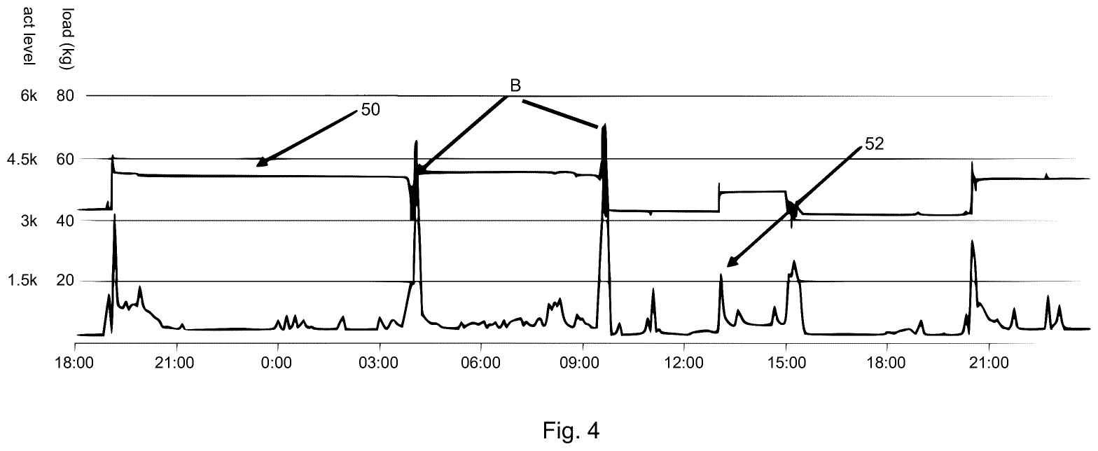

FIG. 4 depicts sample sensor data from the load cells 14 taken over a period of time from the bed 4 of the patient 2 (i.e. who is bedridden and is unable to roll and/or reposition himself or herself regularly). The patient 2 would require assistance in diaper changes. The upper line in FIG. 4 is a measure of changes in apparent bed weight 50 over time, while the lower line is a measure of the movement strength 52. The peaks B in FIG. 4 at approximately 4:00 and 9:00 in both the upper and lower lines may be indicative of the likely diaper change 54.

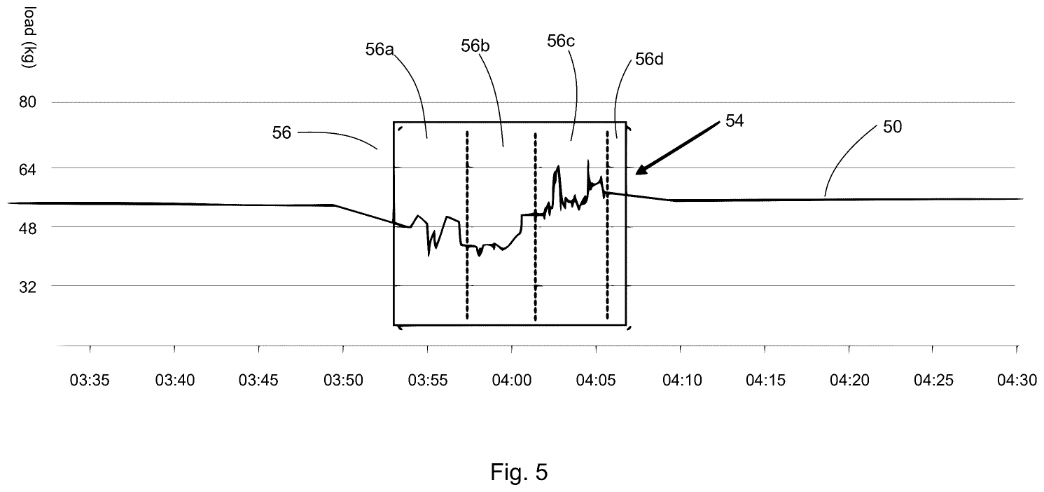

FIG. 5 depicts a portion of the sensor data from FIG. 4 over the period of time between approximately 3:30 and 4:30. In particular, only the data corresponding to the apparent bed weight 50 (i.e. the upper line in FIG. 4) is depicted. The data shown in FIG. 5 shows the data for a typical pattern that may be associated with the likely diaper change 54. The pattern is further divided into four diaper change portions 56 (i.e. 56a, 56b, 56c, 56d), with each of the diaper change portions 56 corresponding generally to a step in the likely diaper change 54. For example, the diaper change portion 56a may correspond to rolling the patient 2 away to one side, the diaper change portion 56b may correspond to replacing the diaper, the diaper change portion 56c may correspond to rolling the patient 2 to the other side and fastening the diaper, and the diaper change portion 56d may correspond to rolling the patient 2 back to a normal sleeping position. All of the four diaper change portions 56 may take place within 10 minutes (i.e. all four of the diaper change portions 56 may be completed within 10 minutes). Although FIG. 5 only depicts data corresponding to the apparent bed weight 50, it is understood that data corresponding to the movement strength 52 may also be classified into the diaper change portions 56.

The sever 16 may be configured to recognize such a pattern (or a similar pattern) in the data regarding the apparent bed weight 50 and/or the movement strength 52 received from the load cells 14 in the future as being indicative of the likely diaper change 54.

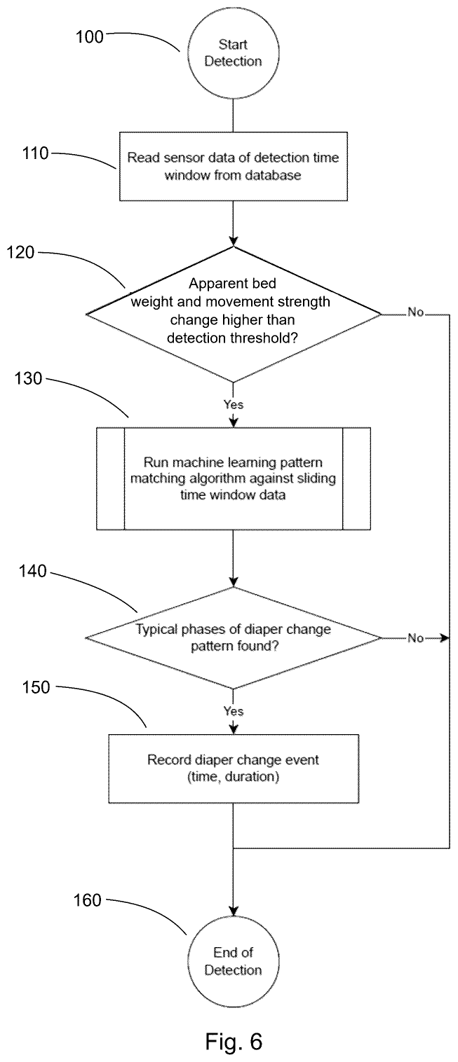

FIG. 6 is a flow chart depicting the process flow of the system 10. Detection is started by the device 12 at step 100. Sensor data (e.g. data regarding the apparent bed weight 50 and data regarding movement strength 52) is collected by the load cells 14 on the device 12 and transmitted by the transmitter 18 to the server 16. The data along with the time that the data was taken or received may be stored in the database 20.

At step 110, the server 16 may be configured to retrieve from the database 20 the sensor data for a particular detection time window.

At step 120, the server 16 is configured to process the data. In particular, the server 16 is configured to determine whether the data regarding the apparent bed weight 50 and/or the data regarding the movement strength 52 is above particular pre-set detection thresholds. If the server 16 determines that the data regarding the apparent bed weight 50 and/or the data regarding the movement strength 52 is below the particular pre-set detection thresholds, the process flow proceeds to step 160 and ends.

If the server 16 determines that the data regarding the apparent bed weight 50 and/or the data regarding the movement strength 52 is above a particular pre-set detection threshold, the process proceeds to step 130. In some embodiments, the server 16 may require that both the apparent bed weight 50 and the movement strength 52 are above the particular pre-set detection thresholds before proceeding to step 130. In other embodiments, the server 16 may require that only one of the apparent bed weight 50 and the movement strength 52 be above the particular pre-set detection thresholds before proceeding to step 130.

At step 130, the server 16 may be configured to apply machine learning techniques to perform one or more pattern matching algorithms against the sliding time window data at step 130.

At step 140, the server 16 is configured to determine, based at least in part on the results of applying the machine learning techniques at step 130, whether the pattern of data corresponding to the typical phases of the diaper change portions 56 is found in the data from the particular detection time window. The data may include one or both of the apparent bed weight 50 and the movement strength 52. If that is the case, at step 150, the server 16 is configured to record in the database 20 that the likely diaper change 54 occurred. Information regarding the approximate time and approximate duration of the likely diaper change 54 may also be recorded in the database 20. The process flow then proceeds to step 160 and ends.

If the server 16 determines that the pattern of data corresponding to the typical phases of the diaper change portions 56 is not found in the data from the particular detection time window, then the process flow proceeds to step 160 and ends.

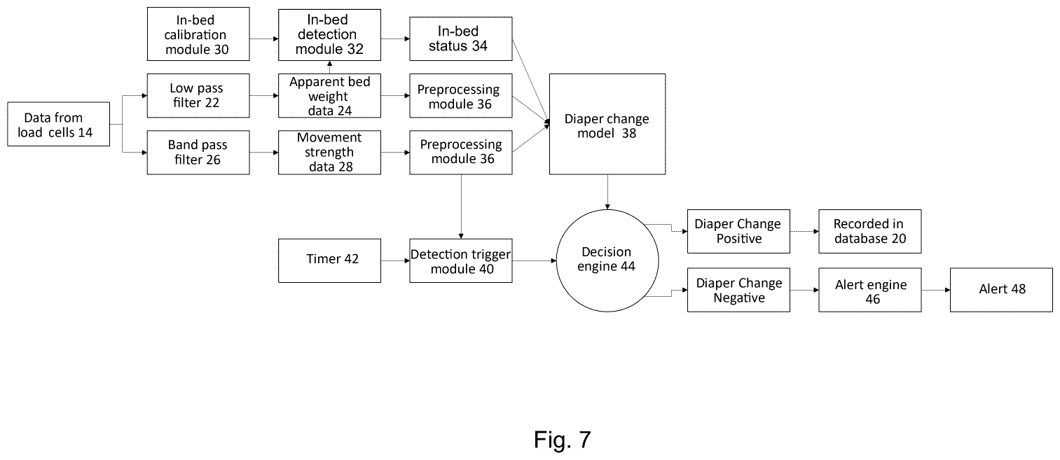

FIG. 7 is a system diagram depicting some of the aspect of the system 10. The load cells 14 in the device 12 may be configured to detect forces exerted on the load cells 14 and convert them into electrical signals, which are transmitted to the server 16. The server 16 may be configured to apply digital signal processing on the electrical signals. For example, the server 16 may apply a low pass filter 22 on the electrical signals to generate apparent bed weight data 24. In addition or alternatively, the server 16 may apply a band pass filter 26 to generate movement strength data 28.

The server 16 may utilize an in-bed calibration module 30 to allow for calibration of the bed 4 before use. The server 16 may then use an in-bed detection module 32 to determine whether the patient 2 may be in the bed 4. The in-bed detection module 32 may use information from the apparent bed weight data 24 to arrive at this determination. For example, when the patient 2 is placed into the bed 4, the apparent bed weight data 24 may show a sustained increased in the apparent bed weight 50. The in-bed detection module 32 may also use information from the in-bed calibration module 30 to assist in determining when the patient 2 is placed in the bed 4 (e.g. to provide a baseline for when the patient 2 is not in the bed 4). The in-bed detection module 32 will trigger an in-bed status 34 (between the patient 2 being shown as in the bed 4 and the patient 2 being shown as not in the bed 4).

The server 16 may utilize a preprocessing module 36 on one or both of the apparent bed weight data 24 and the movement strength data 28. The preprocessing module 36 applies preprocessing techniques on the data to transform it into a relatively clean, normalized time series that may be more helpful for subsequent machine learning steps. For example, the preprocessing module 36 may employ one of more of the following: segmentation, interpolation, noise reduction, normalization, and feature extraction.

Segmentation involves segmenting the time series data into fixed-length windows that represent possible diaper change events. This helps to focus subsequent machine learning steps on smaller, more relevant segments of data.

With respect to interpolation, the data collected may be at irregular intervals (as described above) or may be missing data. Interpolation generates values at regular time intervals (e.g. 1 sample per second), which may be required or helpful for subsequent machine learning steps.

Noise reduction applies a moving average to reduce any significant variability that may be introduced during interpolation. This may allow for a more robust and consistent pattern detection when used on other patients and beds.

Normalization makes the data comparable across different ones of patients and beds by removing the influence of absolute weight differences.

Feature extraction involves extracting features like weight variability and peak detection to help in subsequent machine learning steps.

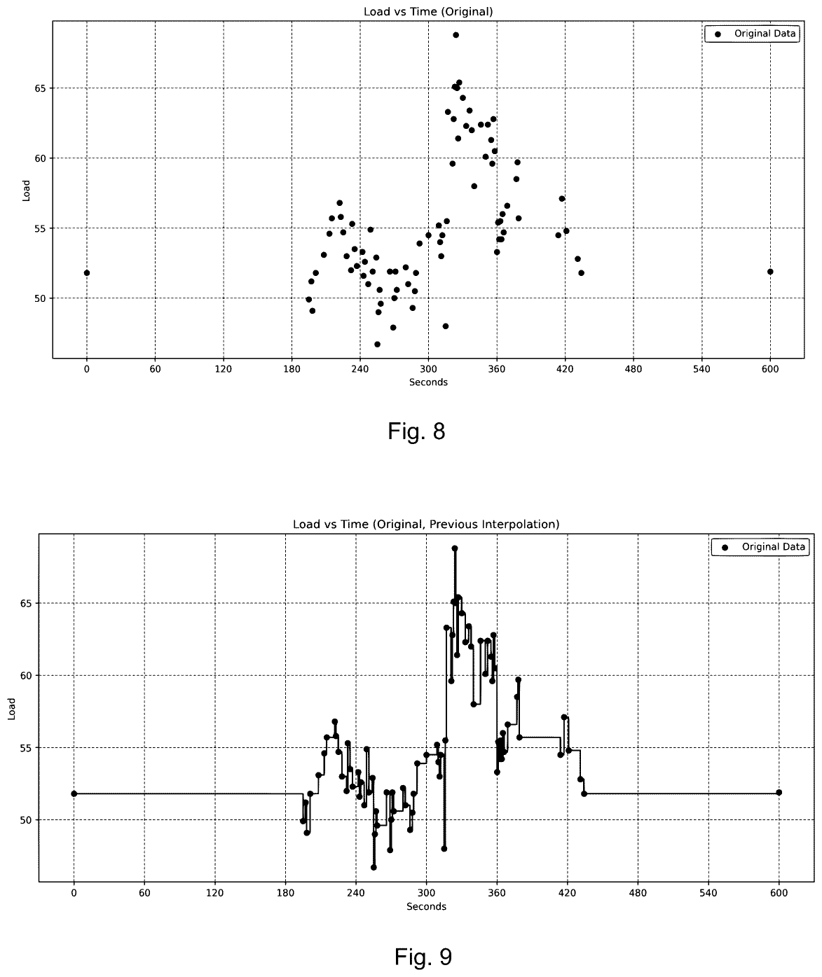

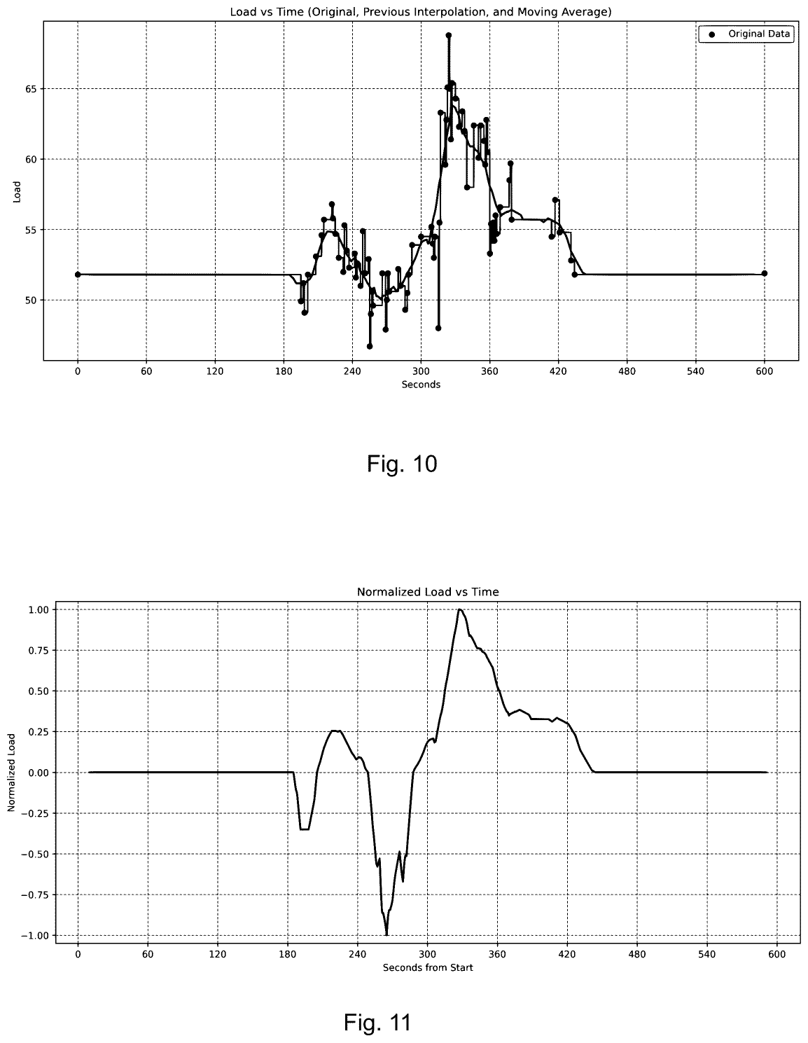

FIGS. 8 to 11 depict examples of preprocessing applied to the data. FIG. 8 depicts raw irregular interval data from the load cells 14. FIG. 9 depicts the data from FIG. 8 with interpolation applied to generate constant interval data. FIG. 10 depicts the data from FIG. 9 with noise reduction applied to generate more smoothed data. FIG. 11 depicts the data from FIG. 10 with normalization applied.

The data from the preprocessing module 36 (from one or both of the apparent bed weight data 24 and the movement strength data 28) and from the in-bed status 34 may be used by a diaper change detection model 38. In some embodiments, if the in-bed status 34 indicates that the patient 2 is not in the bed 4, the diaper change model 38 may not proceed with diaper change detection. In such cases, any changes in the apparent bed weight 50 and/or the movement strength 52 may be due to staff working on the bed 4.

The server 16 may utilize a detection trigger module 40 to determine when a detection should be made. This determination may be based, at least in part, on a timer 42 that will cause the detection to be made at regular time intervals. This determination may also be based, at least in part, on the data from the movement strength data 28. For example, the detection may be made when the movement strength data 28 indicates a possible movement of the patient 2.

When the detection trigger module 40 determines that a detection should be made, the diaper change detection model 38 inputs the data to a decision engine 44. The decision engine 44 may be configured to determine whether the likely diaper change 54 has occurred. If the decision engine 44 determines that the likely diaper change 54 has occurred, information regarding the likely diaper change 54 is recorded in the database 20. Alternatively, if the decision engine 44 determines that the likely diaper change 54 has not occurred during a pre-set period of time (e.g. 6 hours), an alert engine 46 may be triggered. The alert engine 46 may cause an alert to be generated.

The machine learning techniques applied by the server 16 may include one or more of the following: (1) early classification; (2) support vector machine (SVM) and global alignment kernel (GAK); and (3) learning shapelets. In some embodiments, the learning shapelets approach is used. In this approach, a collection of shapelets that linearly separate the time series measurements is learned. The dot product of learned shapelet and measurement window is computed to find repositioning events. The learning shapelets approach may work well on even a small set of training data. It has been found that this approach is able to achieve a correct classification rate of 91%.

The goal of shapelet discovery is to find short, discriminative subsequences (called shapelets) in the time series that are characteristic of a specific class or event. These shapelets may assist in distinguishing between different classes (e.g. detecting a diaper change versus no diaper change).

A typical process may involve the following: (1) extracting a plurality of subsequences of different lengths from the time series; (2) calculating the distance between the subsequences and the entire time series; (3) scoring and selecting subsequences (shapelets) based on how well they separate different classes (e.g. using metrics such as information gain); and (4) outputting the most discriminative shapelets that can serve as features for the classification task.

For detection of possible diaper change events, the shapelet discovery may identify patterns in the apparent bed weight data 24 (e.g. a weight shift) that consistently occurs during the rolling of the patient 2. In some embodiments, the shapelet discovery may use a 20-second period.

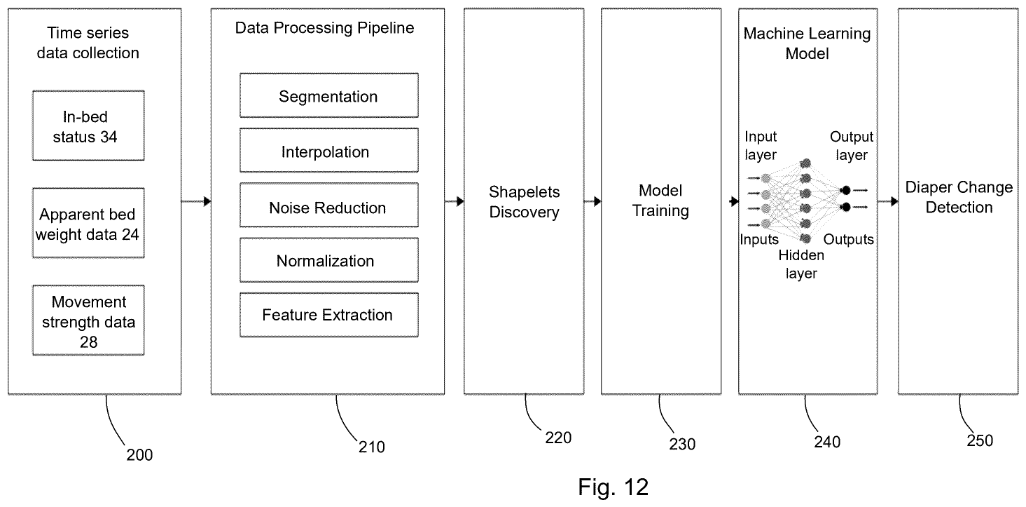

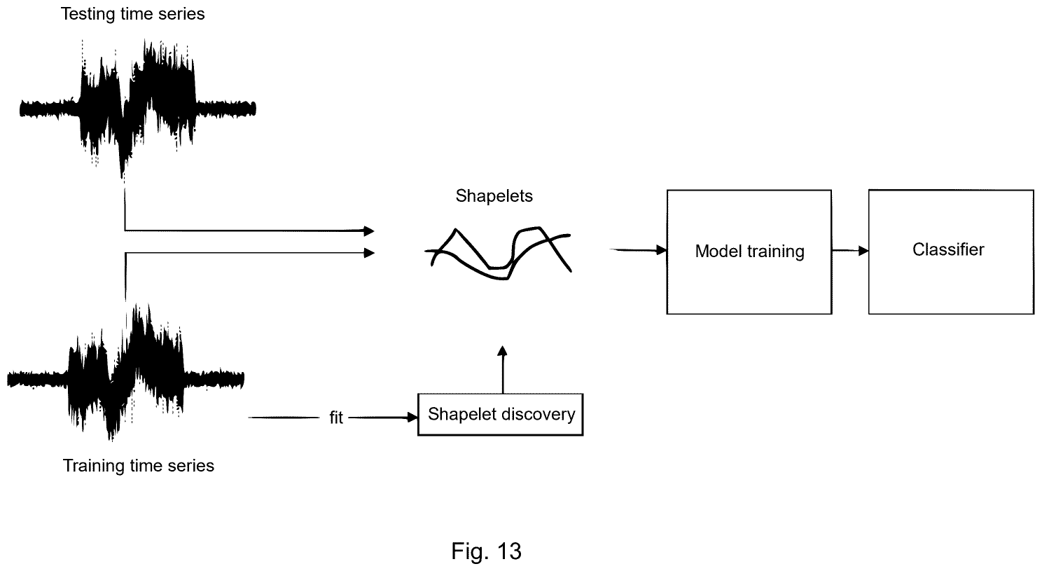

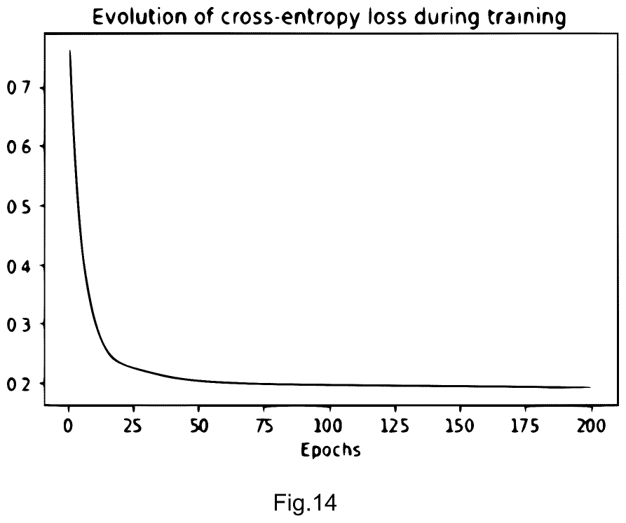

FIG. 12 depicts a possible machine learning detection engine data processing workflow. FIG. 13 depicts a possible shapelet discovery workflow. FIG. 14 depicts a plot of loss over total training iterations.

Referring to FIG. 12, at step 200, three inputs may be used for the process: (1) the in-bed status 34; (2) the apparent bed weight data 24; and (3) the movement strength data 28. At step 210, the data may undergo preprocessing (i.e. one or more of segmentation, interpolation, noise reduction, normalization, and/or feature extraction). At step 220, shapelet discovery is carried out. At step 230, model training is carried out. At step 240, a machine learning model is applied. At step 250, the diaper change detection model 38 is ready for use.

Referring to FIG. 13, in order to train the machine learning model, previous data (include data on known diaper changes) may be collected and inputted into the machine learning model. In some embodiments, 70% of the data may be used for training, while 30% of the data may be used for testing and verification. The classifier may be the output of the training.

FIG. 14 provides an indication of the effectiveness of the diaper change detection model 38.

Using the above techniques, the server 16 may be configured to determine whether the likely diaper change 54 has occurred. The data regarding the likely diaper change 54 may be recorded by the server 16 in the database 20 to ensure that the patient 2 is receiving regular diaper changes.

It is understood that the system 10 may be used in different settings. In some embodiments, the patient 2 may be bedridden (e.g. such as in a hospital or an assisted living facility). However, in other embodiments, the system 10 may also be used in home settings.

It will be appreciated by those skilled in the art that the preferred embodiment has been described in some detail but that certain modifications may be practiced without departing from the principles of the invention.

1. A method for detecting a possible changing of a diaper of a patient in a bed comprising one or more bed supports, the method comprising the steps of:
providing a device comprising one or more load cells and a transmitter;
placing the device under one of the one or more bed supports;
sensing, by the one or more load cells, of a magnitude of a force exerted on the one or more load cells at each of a plurality of times over a period of time, the magnitude of the force corresponding to an apparent bed weight;
transmitting, by the transmitter to a server, data sensed by the one or more load cells, the data comprising information corresponding to a magnitude of the force exerted on the one or more load cells at each of the plurality of times;
storing, by the server, of the data to generate a record comprising a temporal history of the magnitude of the force exerted on the one or more load cells over the period of time;
configuring the server to recognize a pattern indicative of the possible changing of the diaper of the patient based, at least in part, on changes in the apparent bed weight;
determining, by the server whether the possible changing of the diaper of the patient has occurred, based, at least in part on whether the temporal history comprises the pattern; and
recording, by the server, a time in the period of time when the temporal history comprises the pattern.

2. The method of claim 1 further comprising the step of:
sensing, by the one or more load cells, of a degree of vibration of the force exerted on the one or more load cells at each of the plurality of times over the period of time, the degree of vibration of the force corresponding to a movement strength.

3. The method of claim 2, wherein the step of configuring the server to recognize the pattern indicative of the possible changing of the diaper of the patient is also based, at least in part, on changes in the movement strength.

4. The method of claim 1, wherein the pattern comprises at least four phases, the at least four phases corresponding to (1) rolling of the patient away to one side; (2) replacing the diaper of the patient; (3) rolling the patient back to another side to fasten the diaper; and (4) rolling the patient back to a resting position.

5. The method of claim 1, wherein the step of configuring the server to recognize the pattern indicative of the possible changing of the diaper of the patient based, at least in part, on changes in the apparent bed weight comprises using shapelet learning techniques.

6. The method of claim 3, wherein the step of configuring the server to recognize the pattern indicative of the possible changing of the diaper of the patient also based, at least in part, on changes in the movement strength, comprises using shapelet learning techniques.

7. The method of claim 1, further comprising the step of:
determining, by the server, whether the patient is likely in the bed at one of the plurality of times, based, at least in part, on the apparent bed weight.

8. The method of claim 1, further comprising the step of:
generating, by the server, of an alert if the server determines that the possible changing of the diaper for the patient has not occurred within a pre-set length of time.

9. The method of claim 1, wherein the step of storing, by the server, of the data to generate the record comprising the temporal history of the magnitude of the force exerted on the one or more load cells over the period of time comprises storing of the data in a database in communications with the server.

10. The method of claim 1, wherein the step of transmitting, by the transmitter to the server, data sensed by the one or more load cells occurs approximately every five minutes when the magnitude of the force exerted on the one or more load cells has not changed within a first pre-set interval of time.

11. The method of claim 1, wherein the step of transmitting, by the transmitter to the server, data sensed by the one or more load cell occurs approximately every second when the magnitude of the force exerted on the one or more load cells has changed within a second pre-set interval of time.

12. The method of claim 1, wherein the step of storing, by the server, of the data to generate the record comprising the temporal history of the magnitude of the force exerted on the one or more load cells over the period of time further comprises processing, by the server, of the data by applying one or more of the following: segmentation of the data, interpolation of the data, noise reduction of the data, normalization of the data, and feature extraction of the data.

13. The method of claim 1, wherein the step of determining, by the server whether the possible changing of the diaper of the patient has occurred, is also based, at least in part on whether the changes in the apparent bed weight are above a first pre-set threshold.

14. The method of claim 3, wherein the step of determining, by the server whether the possible changing of the diaper of the patient has occurred, is also based, at least in part on whether the changes in the movement strength are above a second pre-set threshold.
